(19) United States
(12) Patent Application Publication
(10) Pub. No.: US 2002/0120231 A1
(43) Pub. Date: Aug. 29, 2002

(54) Subcutaneous injection set with secondary injection septum

(76) Inventors: Joel S. Douglas, Groton, CT (US); Robert L. Hugo, Gilroy, CA (US); Cynthia Zhang, Saratoga, CA (US)

Correspondence Address: T. Gene Dillahunty, BURNS, DOANE, SWECKER & MATHIS, L.L.P., P.O. Box 1404, Alexandria, VA, 22313-1404, US

(21) Appl. No.: 10/076,907

(22) Filed: Feb. 13, 2002

Related U.S. Application Data
(63) Continuation-in-part of application No. 09/675,159, filed on Sep. 29, 2000.
(63) Continuation-in-part of application No. 09/933,452, filed on Aug. 20, 2001.
(60) Provisional application No. 60/271,079, filed on Feb. 26, 2001.
(60) Provisional application No. 60/195,702, filed on Apr. 7, 2000.
(60) Provisional application No. 60/188,624, filed on Mar. 13, 2000.
(60) Provisional application No. 60/176,538, filed on Jan. 18, 2000.
(60) Provisional application No. 60/250,264, filed on Nov. 29, 2000.

(51) Int. Cl. A61M 37/00

(52) U.S. Cl. 604/82; 604/284

(57) ABSTRACT

An infusion system, comprising a housing; and a connecting hub which is attachable to the housing, the connecting hub having an internal Y-shaped flow channel structure which comprises a first flow channel exiting the distal end of the connecting hub; a second flow channel exiting the proximal end of the connecting hub; and a third flow channel exiting the proximal end of the connecting hub, the first, second and third flow channels all intersecting within the connecting hub.

### RELATED CASES

The present application claims the benefit of priority under 35 U.S.C. Section 119 to U.S. provisional patent application Ser. No. 60/271,079, filed Feb. 22, 2001, and is a is a continuation-in-part of U.S. patent application Ser. No. 09/675,159, which claims the benefit of priority under 35 U.S.C. Section 119 to U.S. provisional patent application Ser. No. 60/195,702, filed Apr. 3, 2000 and No. 60/188,624 filed Mar. 13, 2000 and No. 60/176,538 filed Jan. 18, 2000, the entire contents of each of which are incorporated by reference herein.

### SUMMARY OF THE INVENTION

In accordance with the present invention, an injection system is provided with the ability to simultaneously supply a continuous or intermittent basal rate from one delivery device and a bolus injection from another delivery device. Thus, the present invention is suited for delivery of medication (or other fluids which use varying basal and bolus dosages).

According to various embodiments, the present invention provides an infusion system, comprising a housing; and a connecting hub which is attachable to the cannula housing, the connecting hub having an internal Y-shaped flow channel structure. In various embodiments, the connecting hub is releasably attached to the cannula housing. The internal Y-shaped flow channel structure comprises a first flow channel adapted to connect to a proximal end of an infusion cannula; a second flow channel adapted to connect to a distal end of an infusion delivery tube; and a third flow channel which is covered by a septum, the first, second and third flow channels all intersecting within the connecting hub.

The connecting hub may be attached to the proximal end of the cannula housing. According to these embodiments, the connecting hub is attached to the proximal end of the cannula housing by a pair of fasteners, wherein each fastener comprises a finger on one of the cannula housing and connecting hub, and a lever on the other of the cannula housing and connecting hub. In various embodiments, the lever is cantilevered.

The present invention also provides a method of infusing two different delivery streams, e.g. medication or monitoring or trace element, into a patient through a single subcutaneous pathway, comprising subcutaneously inserting a distal end of an infusion cannula into a patient, the infusion cannula being supported by a housing at its proximal end; attaching a connecting hub to the housing, the connecting hub having an internal Y-shaped flow channel structure comprising first, second and third flow channels which intersect within the connecting hub; introducing a first delivery stream through a delivery tube and into the second flow channel; and introducing a second delivery stream through a septum and into the third flow channel, the second and third flow channels intersecting into the first flow channel such that the first and second delivery streams pass together out of the first flow channel, passing into the tissue through the housing and infusion cannula. According to the embodiments of the present invention, the second delivery stream is injected through the septum by a delivery device such as a syringe. Thereafter, the connecting hub may be disconnected from the housing; and a plugging system attached to the cannula housing while leaving the distal end of the infusion cannula within the tissue.

According to an embodiment, the present invention provides an infusion device which minimizes the volume remaining in the infusion indwelling catheter so essentially all the volume of the secondary injection is delivered to the target area, e.g. tissue.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to the embodiments of the apparatus, method and systems, given only by way of example, and with reference to the accompanying drawings, in which:

FIGS. 1 and 2 are top plan views, with portions broken away in FIG. 2, of an infusion system in accordance with the present invention;

FIG. 3 is a top plan view of a cannula housing of the system illustrated in FIG. 2;

FIG. 4 is a front elevation view of the housing of FIG. 3;

FIG. 5 is a rear upsidedown elevation view of the housing of FIG. 3;

FIG. 6 is a left side elevation view of the housing of FIG. 3;

FIG. 7 is a cross-sectional view, taken at line 7-7 in FIG. 3;

FIGS. 8 and 9 illustrate enlarged views of portions of the cannula housing of FIG. 3;

FIG. 10 is a top plan view of the connecting hub of the system of FIG. 1;

FIG. 11 is a side elevation upsidedown view of the connecting hub of FIG. 10;

FIG. 11a is a side elevation view of a positioning block needle hub of the device in FIG. 10;

FIG. 12 is a front elevation view of the device in FIG. 10;

FIG. 13 is a top plan view of an insertion device for use with the present invention;

FIG. 14 is a side elevation view of the insertion device of FIG. 13;

FIG. 15 is a top plan view of a plugging system for use with the present invention;

FIG. 16 is a front elevation view of the plugging system of FIG. 15;

FIG. 17 is a cross-sectional view taken at line 17-17 in FIG. 16;

FIG. 18 is a longitudinal cross-sectional view of the soft infusion cannula of FIG. 1.

FIG. 19 is a sectional side elevation view of the infusion system of FIGS. 1 and 2.

### DETAILED DESCRIPTION OF THE DRAWINGS

Before the present articles and methods are disclosed and described, it is to be understood that the terminology used herein is for the purposes of describing particular embodiments only and is not intended to be limiting. It must be noted that, as used in the specification and appended claims, the singular forms “a”, “an” and “the” include plural referents unless the context clearly dictates otherwise.

The present invention provides a disposable injection system that provides the use with the ability to infuse from a pump or syringe independently (depending on the type or quantity of substance desired). According to embodiments, such delivery may be accomplished subcutaneously without disconnecting the infusion catheter. Moreover, the present system accomplishes this without compromising the sterility of medication or other delivery substance of choice to being continuously or intermittently infused from a main reservoir. Referring to the drawing figures, like reference numerals designate identical or corresponding elements throughout the several figures.

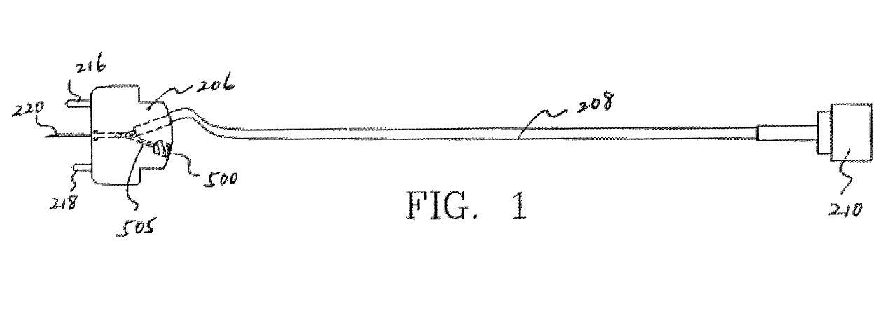

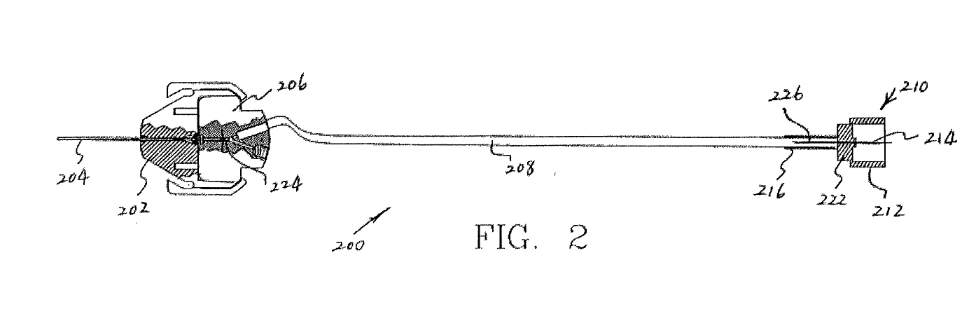

FIGS. 1 and 2 show top plan views (with portions broken away in FIG. 2) of a medication infusion system according to the present invention. Infusion system 200 includes a housing 202 and connecting hub 206. Housing 202 is adapted to hold a soft (indwelling) cannula 204 for positioning subcutaneously in a patient.

Connecting hub 206 is positioned to the proximal end of the housing 202. A length of tubing 208, flexible according to certain embodiments, is secured to the proximal portions of connecting hub 202. A proximal connector 210 is secured to the proximal end of the tubing 208. Proximal connector 210 may be select from connectors which are compatible with the connector of the primary infusion device such as a luer connector.

In accordance with the present invention, connecting hub 206 has an internal Y-shaped flow channel structure (as seen in FIG. 10). As will be explained in detail herein, the internal Y-shaped flow channel structure permits simultaneous delivery, e.g. medication from two different sources (for example: a catheter and a syringe). According to certain embodiments, a first substance is delivered through a primary infusion device, infusing through tubing 208, with a second medication delivered by way of a syringe piercing septum 500. The internal Y-shaped flow channel structure of connecting hub 206 permits simultaneous subcutaneous delivery to the patient through soft cannula 204 (of housing 202).

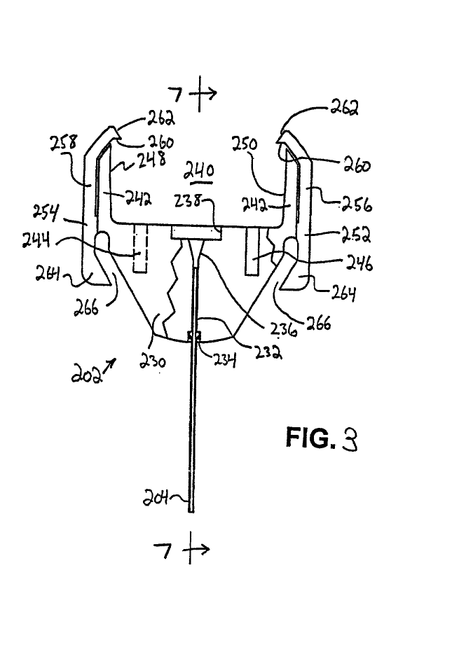

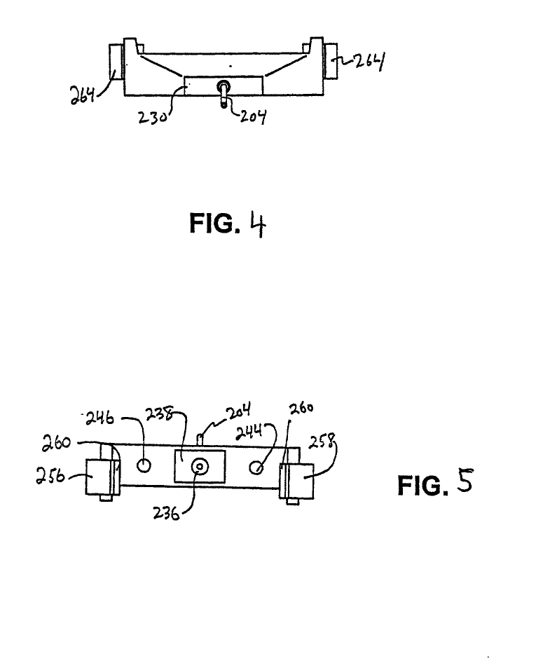

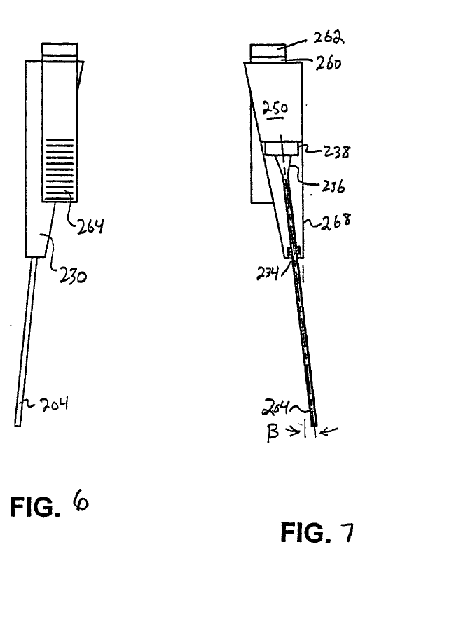

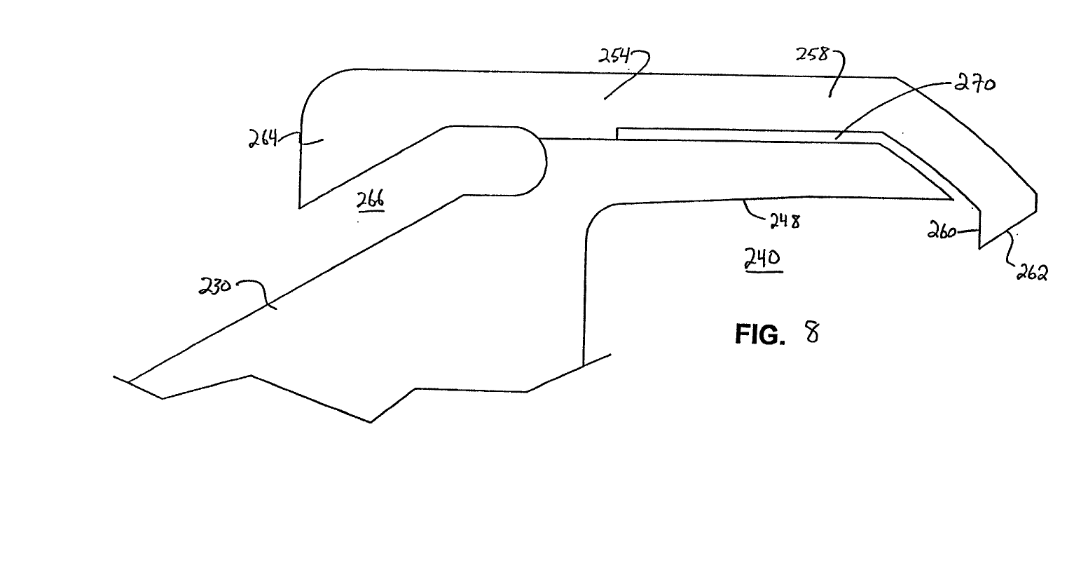

As shown in FIG. 1, connecting hub 206 comprises a pair of distally extending pins 216, 218 which are inserted into and mate with complementarily placed and configured holes or bores 244, 246 (FIG. 3) in housing 202.

Further details of housing 202 are shown in FIGS. 3 to 9, as follows.

Housing 202 preferably includes a generally frustoconical shaped, distally extending portion 230, through which a bore 232 extends. Depending on the embodiment, bore 232 may be widened at its distal end to form a cannula recess 234, for reasons which will be discussed further below. The proximal end of the bore 232 widens in a frustoconically shaped proximal portion 236. Thus, the proximal portion 236 is tapered in order to guide a tube, such as tube 220, when inserted into the bore 232 through proximal opening 238. Housing 202 may also include blind bores for receiving the pins from the connecting hub. For example, blind bores 244, 246 may be sized and positioned to receive pins 216 and 218 (FIG. 1). It is to be understood that other pin-bore designs are possible, and that any number of pins and bores are within the spirit and scope of the present invention. Conversely, using more than two pin-bore combinations may provide better registration between the hub and the housing.

The proximal end of housing 202 may also include a pair of guide extensions 242, with one on each lateral side of the housing, which together delimit an open proximal space 240 sized and shaped to receive portions of the connecting hub 206. Each guide extension 242 includes a laterally inwardly directed, slightly tapered guide surface 248, 250. The guide surfaces 248, 250 are sized and oriented so that they provide initial guidance to the outer surfaces of connecting hub 206 when connecting hub 206 and housing 202 are brought together, described in greater detail below.

Housing 202 may also include at least one, but more preferably two, fasteners 252, 254, which are positioned on its lateral sides. Fasteners 252, 254 are provided to fasten connecting hub 206 to housing 202 when the hub and housing are properly mated together (see FIG. 2). Fasteners 252, 254 may include releasable fingers 256, 258 which extend proximally from housing 202. Fingers 256, 258 may include a bearing surface 260 which is distally directed for bearing against portions of the hub 206 which face proximally (again, see FIG. 2). Adjacent to the bearing surface(s) 260, each finger 256, 258 is provided with an angled cam surface 262. Cam surfaces 262 advantageously provide a place for connecting hub 206 to bear against fingers 256, 258 and push them laterally outward, to thereby spread fingers 256, 258 and thus permit connecting hub 206 to enter space 240 when moved distally relative to the housing 202.

Fasteners 252, 254 may also include levers 264 on their proximal ends, positioned opposite the fingers 256, 258. Levers may be cantilevered. Levers 264 are positioned laterally spaced from the housing 202 so that there are spaces 266 between the levers and the housing into which the levers can be flexed. Flexure of the levers 264 laterally inward cause the fingers 256, 258 to flex laterally outward, thus permitting the connecting hub 206 to enter and exit the space 240 more easily. As will be readily appreciated by one of ordinary skill in the art, pulling proximally on the connecting hub 206 (when it is positioned in the space 240) with sufficient force will also spread fingers 256, 258 apart such that connecting hub 206 becomes released from the housing 202 and fasteners 252, 254.

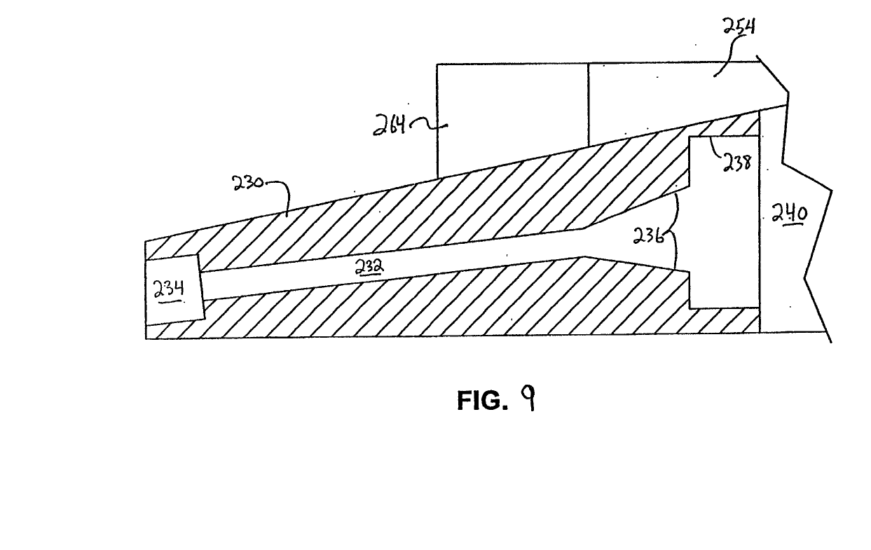

As illustrated in FIGS. 4, 6, and 7, soft cannula 204 may optionally be angled relative to the bottom surface 268 of the housing 202 at an angle β, wherein β can be any acute angle.

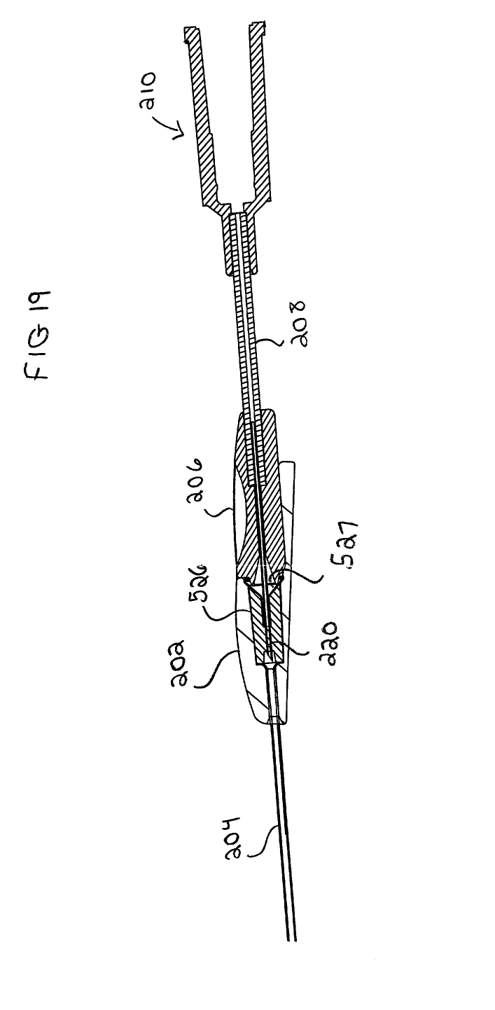

FIGS. 8 and 9 illustrate enlarged views of portions of connecting hub 206. Specifically, FIG. 8 illustrates fasteners 254, including the channel 270 between the finger 258 and the housing 202. FIG. 9 illustrates the bore 232, without the soft cannula 204 positioned in the bore. The counter bore formed from 238 and 236 may also hold a seal 525 which can be glued or staked in place instead of being located on needle 220. Alternatively, as shown in FIG. 19, a septum 526 can also be received in housing 202. Optionally, septum 526 can be held in position by a funnel shaped metal guide 527 which orients hollow tube 220 for passage through the center of septum 526.

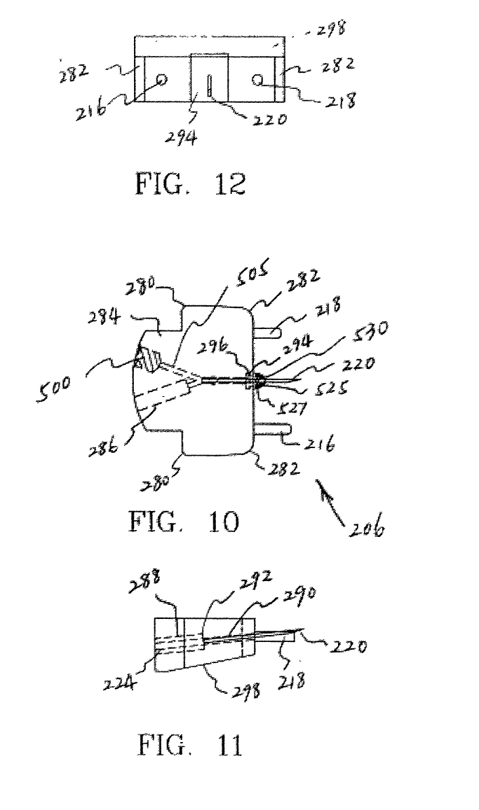

FIGS. 10 to 12 illustrate further details of connecting hub 206 with FIG. 10 being a top plan view; FIG. 11 being a side elevation (upsidedown) view; FIG. 11a showing a positioning block needle hub for use with the connecting hub; and FIG. 12 being a front elevation view of the connecting hub. As discussed above, pins 216, 218 and hollow tube 220 extend distally from connecting hub 206. Connecting hub 206 includes surfaces 280 which are oriented and positioned so that bearing surfaces 260 of fingers 256, 258 (FIG. 3) will bear against surfaces 280 when connecting hub 206 is positioned within space 240. According to various embodiments, cam surfaces 282 are provided on the distal end of connecting hub 206 to bear against cam surfaces 262 on fingers 256, 258, as described above.

A distally extending hollow tube 220 extends from the connecting hub 206, having proximal portion 224 which is in fluid communication with tubing 208. The proximal portion 224 of tube 220 can be secured into connecting hub 206 in any suitable manner, including epoxy or solvent bonding. Hollow tube 220 may be sharpened at its distal most end, e.g., be a needle, or can alternatively it can be blunt.

As seen in FIG. 10, connecting hub 206 has an internal Y-shaped flow channel structure, which permits simultaneous delivery of substances from two different sources, as follows. First, the proximal portion 284 of connecting hub 206 is adapted such that a distal end of tubing 208 may be attached thereto. Specifically, the distal end of tubing 208 may be received into a bore 286 in connecting hub 206. Bore 286 is in fluid communication with flow channel 290 which exits from the distal end of connecting hub 206. Flow channel 505 (which exits from proximal portion 284 of connecting hub 206) forms the second branch of connecting hub 206's internal “Y-shaped” flow structure. Specifically, channel 505 is in fluid communication with bore 286 and flow channel 290. Thus, together, flow channels 286, 505 and 290 form the present invention's internal Y-shaped flow channel system.

It is to be understood that the above embodiment is merely exemplary in that the present invention also includes optional embodiments having more than two flow channels (e.g.: similar to flow channels 286 and 505) which are in fluid communication and “merge” into a single flow channel (e.g.: similar to flow channel 290). Thus, the present invention also includes optional embodiments in which substance delivery from three or more delivery sources can be delivered to the tissue target area through a single passageway. Thus, the present reference to a “Y-shaped” flow channel structure specifically includes all embodiments of the design which have at least two flow channels merging into a single flow channel for delivery to a tissue target region. Thus, the present “Y-shaped” flow channel structure includes “Y's” having more than two flow channels merging into a single flow channel.

It is also to be understood that optional embodiments of the present invention include both of the flow channels which are received into connecting hub 206 being similar to the bore 286/tubing 208 embodiment, or alternatively being similar to the septum 500/flow channel 505 embodiment. Thus, the present invention includes embodiments in which substance delivery is achieved by two tubes (similar to tubing 208) being received into separate flow channels in connecting hub 206. Also, the present invention includes embodiments in which substance delivery is achieved by two syringes being injected into separate flow channels in the connecting hub (passing through separate septums similar to septum 500).

In embodiments of the present invention, proximal end 224 of tube 208 is received into bore 286. Tubing 208 may be glued or attached by other suitable permanent or non-permanent approaches into bore 286 of connecting hub 206. Optionally, bore 286 may include a shoulder 292 where it narrows significantly for connection into flow channel 290. Thus, the internal diameter of bore 286 may be larger than the internal diameter of flow channel 290 according to the embodiments. In addition, the internal diameter of flow channel 505 may also be kept small. According to its embodiments, the present invention allows for minimal fluid volume “dead space” within connecting hub 206. For example, in optional preferred aspects, the volume of flow channel 505 very small, being less than 100 microliters. Optionally, as well, the inner diameter of bore 286 is slightly smaller than the outer diameter of tubing 208, so that tubing 208 is slightly radially compressed, and thus held in place, when received therein. Bore 286 may also have a slightly tapered inner diameter, so that the radial compression of tubing 209 is greatest towards shoulder 292.

In accordance with the present invention, a septum 500 is positioned to cover channel 505. The user can thus simply manually insert a syringe through septum 500 to deliver a secondary stream of substance subcutaneously (in addition to the first stream of medication concurrently being delivered through tubing 208).

In optional aspects, a seal 525 may be received around tube 220. According to its embodiments, seal 525 has an inner restriction surface 530 which seals around tube (e.g.: needle) 220, and an outer surface 527 which can be positioned to form a seal against tapered surface 236 of bore 232 when the connecting hub 206 is received into the housing 202. Seal 525 may alternatively be positioned in cannula housing portion 230, thus forming a septum (septum 526 in FIG. 19). For example, seal 525 could be positioned in housing 202 adjacent taper 236 or proximal opening 238.

As shown in FIG. 11a, in optional embodiments, a positioning block 294 may be received into distal portion 296 of bore 286. Positioning block 294 may comprise a needle hub. Positioning block 294 aligns hollow tube 220 for placement within bore 232 of housing 202. According to an embodiment, positioning block 294 can have a beveled side 300 to assist in aligning the tube 220 at the same angle β as the soft cannula 204. Also, as illustrated in FIG. 11, connecting hub 206 can optionally have a slanted top surface 298.

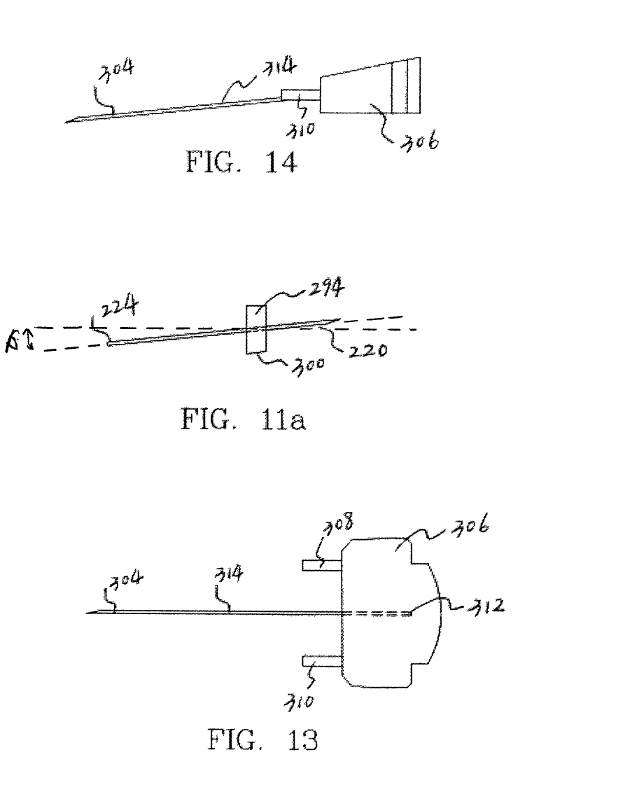

FIGS. 13 and 14 are top plan and side elevation views, respectively, of an insertion device 302 which can be used to initially subcutaneously insert soft cannula 204 into a patient (i.e.: prior to attaching connecting hub 206 to cannula housing 204). Insertion device 302 includes a needle or stylet 304 extending distally from an insertion handle 306. The insertion handle 306 may be similar to the shape and size as the hub 206, and may include a pair of pins 308, 310, operating substantially similar to pins 216, 218. The needle or stylet 304 is of a length such that its sharpened distal end extends beyond the distal end of the soft cannula 204 when insertion handle 306 is mounted onto housing 202 (in the same manner as the connecting hub 206 is later mounted to housing 202 as shown in FIG. 2). The proximal end of needle or stylet 304 is received within handle 306 according to embodiments. In other embodiments, needle 304 can include a score line 314 along its length so that the needle can be broken off and used as an infusion needle. In this optional design, handle 306 would then require a lumen similar to bore 286 in FIG. 10.

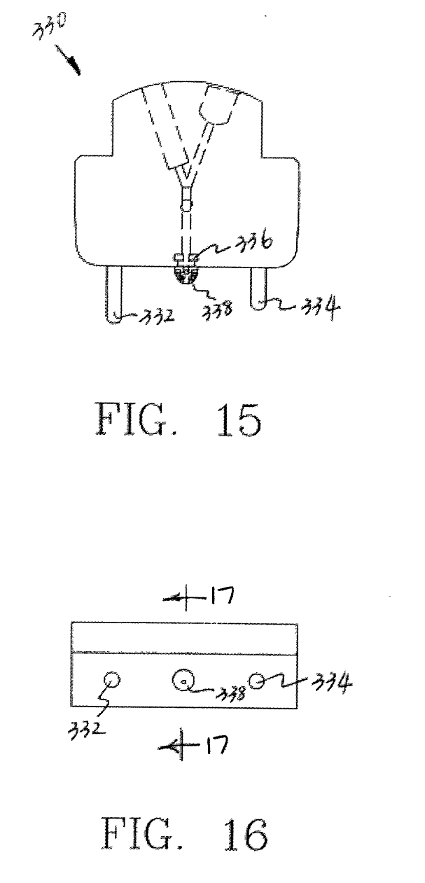

FIGS. 15 to 17 illustrate a plug system 330 for sealing or plugging channel 290 at when housing 202 is positioning soft cannula 204 subcutaneously, and when connecting hub 206 is not attached to housing 202. For example, plug system 330 can be conveniently used when connecting hub 206 is periodically removed. An advantage of plugging system 330 is that it prevents contamination while soft (indwelling) cannula 204 remains in position subcutaneously in the tissue or target. Thus, plugging system 330 can be used to seal the bore 232 in housing 202, and therefore restrict access into the tissue/target by dirt and pathogens or unwanted contaminants/interferents (when connecting hub 206 is disconnected from the housing 202 and replaced by plugging system 330), as follows. Plug 338 may alternatively be positioned within housing portion 230, thus forming a septum. For example, plug 338 could be positioned in housing 202 adjacent taper 236 or proximal opening 238 (e.g. see septum 526 in FIG. 19).

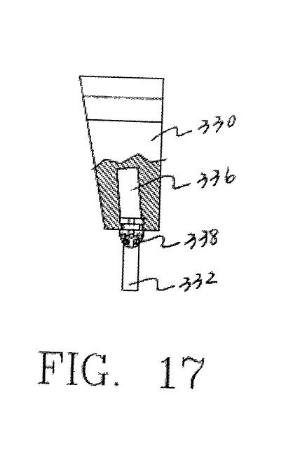

FIG. 15 is a top plan view of plugging system 330; FIG. 16 is a front elevation view of plugging system 330, and FIG. 17 is a cross-sectional view taken at line 17-17 in FIG. 16. The outside size and shape of plugging system 330 may be the same or similar to the insertion handle 306 and the connecting hub 206, described above. Plugging system 330 may include a pair of pins 332, 334 to mate with blind bores 244, 246, as described above. Plugging system 330 also includes a central plugging element 336 which may have a conical, frustoconical, or other such similar tip 338 extending distally therefrom. According to embodiments of the present invention, plugging element 336 may be formed of a somewhat soft material which is somewhat compressible, so that when the plug 330 is mounted to housing 202 (in the same manner as connecting hub 206 and handle 306) plugging element 336 will enter into and seal the proximal portions of bore 232. By way of example and not of limitation, element 338 can be formed of a medical grade silicon rubber. According an optional aspect of the present invention, plugging element 336 may be formed from a material which is doped with an antimicrobial or similar compound to inhibit the growth of microorganisms on the plugging element itself or on the surfaces against which it seats.

The size and taper of plugging element 336 and its tip 338 may be selected to plug and seal bore 232 in housing 202 according to an embodiment. In various embodiments, the size and taper of plugging element 336 and its tip 338 can be selected to seal and plug the bore 232 at the junction of bore 232 and taper 236, or the junction of taper 236 and proximal opening 238, or opening 238 itself, or any combination of these structures by forming plugging element 336 with a complementary shape. (See FIG. 9).

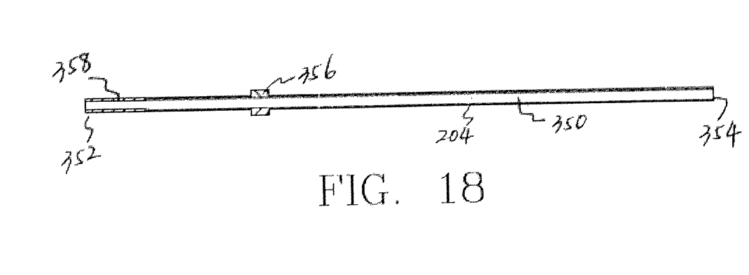

FIG. 18 illustrates a cross-sectional view of soft cannula 204 (which projects from housing 202 as seen in FIGS. 3 to 7). The soft cannula 204 includes a longitudinally extending lumen 350, which extends between a proximal end 352 and a distal end 354. A positioning and retention bead, ring, or lug 356 is formed on the outer surface of the cannula 204, and is sized to fit into recess 234 at the distal end of housing 202. In one aspect of the invention, bead 356 is sized relative to the recess 234 so that bead 356 can securely hold soft cannula 204 in housing 202. In another aspect of the present invention, bead 356 assists in positioning the distal end 354 of cannula 204 at a predetermined distance from housing 202. In yet another aspect of the present invention, cannula 204 is permanently or non-permanently mounted in bore 232 by any of numerous ways. For example, cannula 204 may be permanently mounted in bore 232 by solvents or adhesive bonding, ultrasonic welding, heat staking, or the like.

In accordance with the present invention, various embodiments of a method are also set forth. The operation of the exemplary embodiment of the present invention described above and illustrated in FIGS. 1 to 18 will now be described with reference thereto.

Initially, housing 202 is mounted to insertion handle 306 so that the distal end of the needle or stylet 304 extends distally from the distal end of soft cannula 204. Needle 304 is then subcutaneously advanced into the target area/tissue such that soft cannula 204 (received thereover) is also subcutaneously advanced into the tissue/target area. Insertion handle 306 is then retracted proximally by pressing laterally inwardly on one or both of the levers 264, which releases insertion handle 306 from housing 202.

Connecting hub 206, with attached tubing 208 and proximal connector 210, is then inserted into space 240, by laterally displacing the fingers 256, 258 on housing 202. Pins 216, 218 are thus received into bores 244, 246, while guide surfaces 248, 250 guide the outer surfaces of connecting hub 206 as the hub moves distally. Fingers 256, 258 then snap over surfaces 280, thereby securely holding connecting hub 206 to housing 202 with hollow tube (e.g.: needle) 220 extending into bore 232 of housing 202. Proximal connector 210 may be connected to tubing 208 by an adhesive which is selected from a set of epoxies or solvent adhesive that are capable of bonding to the soft tubing.

Medication infusion is then performed, as described above where the embodiments deliver a medication as its substance. Specifically, a first medicated stream may be administered (passing into the patient sequentially through tube 208, connecting hub 206, cannula housing 204 and soft cannula 204). A second medicated stream may also be administered by way of a syringe piercing through septum 500, and entering channel 505 (and then passing sequentially through connecting hub 206, cannula housing 204 and soft cannula 204). The second medicated stream may then be administered concurrently with the first medicated stream. Alternatively, the second medicated stream may be administered alone, without having to remove tube 208 (which delivers the first medicated stream).

Thereafter, connecting hub 206 can be removed when desired by pressing on the levers 264, as described above, to release the hub 206 from housing 202, while pulling hub 206 proximally. Plugging system 330 can then be installed in place of hub 206, as described above. For example, with plugging element 338 forming a seal with one of the proximal structures of housing 202. Thus, housing 202 and cannula 204 can conveniently be left in place on the tissue/target area for later subcutaneous infusion, while the sterility of the site on the patient is enhanced or maintained.

In optional aspects, the device which delivers medication to connecting hub 206 can be (but is not limited to) any of numerous such devices which receive a medication ampule or reservoir, including “pen” type injectors, programmable medication pumps, and those described in U.S. provisional patent application serial No. 60/156,535, filed Sep. 29, 1999, U.S. application provisional patent serial No. 60/170,570, filed Dec. 13, 1999, U.S. provisional patent application serial No. 60/177,762, filed Jan. 24, 2000, and U.S. non-provisional patent application Ser. No. 09/672,103, filed Sep. 29, 2000, the entire contents of each of which are incorporated by reference herein.

While the invention has been described in detail with reference to preferred embodiments thereof, it will be apparent to one skilled in the art that various changes can be made, and equivalents employed, without departing from the scope of the invention.

What is claimed is:

1. An infusion system, comprising:
a housing; and
a connecting hub which is attachable to the housing, the connecting hub having an internal Y-shaped flow channel structure.

2. The system of claim 1, wherein the internal Y-shaped flow channel structure comprises:
a first flow channel adapted to connect to a proximal end of an infusion cannula;
a second flow channel adapted to connect to a distal end of an infusion delivery tube; and
a third flow channel which is covered by a septum, the first, second and third flow channels all intersecting within the connecting hub.

3. The system of claim 2, wherein the first flow channel exits from a distal end of the connecting hub.

4. The system of claim 2, wherein the second flow channel exits from a proximal end of the connecting hub.

5. The system of claim 2, wherein the third flow channel exits from a proximal end of the connecting hub.

6. The system of claim 2, further comprising:
an infusion cannula received into the distal end of the housing.

7. The system of claim 2, wherein the second flow channel is adapted to receive a distal end of the infusion delivery tube therein.

8. The system of claim 1, wherein the connecting hub is attached to the proximal end of the housing.

9. The system of claim 8, wherein the connecting hub is attached to the proximal end of the housing by a pair of fasteners.

10. The system of claim 9, wherein each fastener comprises a finger on one of the housing and connecting hub, and a cantilevered lever on the other of the housing and connecting hub.

11. The system of claim 2, wherein the connecting hub further comprises:
a hollow tube being received into the first flow channel and projecting from the distal end of the connecting hub, the hollow tube being dimensioned to be received within a channel passing through the housing.

12. The system of claim 11, where the channel passing through the housing is tapered.

13. The system of claim 2, wherein the volume of the third flow channel is less than 100 microliters.

14. The system of claim 1, further comprising:
at least one pin or bore on the distal end of the connecting hub, and at least the other of the pin or bore on the proximal end of the housing, the at least one pin being receivable into the at least one bore when the housing and the connecting hub are connected together.

15. The system of claim 4, further comprising:
an infusion delivery tube in fluid communication with the second flow channel.

16. The system of claim 15, wherein the infusion delivery tube is received into the second flow channel.

17. A method of infusing two different medication streams into a target area/tissue through a single subcutaneous pathway, comprising:
inserting a distal end of an infusion cannula into a target area/tissue, the infusion cannula being supported by a housing at its proximal end;
attaching a connecting hub to the housing, the connecting hub having an internal Y-shaped flow channel structure comprising first, second and third flow channels which intersect within the connecting hub;
introducing a first substance stream through a delivery tube and into the second flow channel; and
introducing a second substance stream through a septum and into the third flow channel, the second and third flow channels intersecting into the first flow channel such that the first and second substance streams pass together out of the first flow channel, passing into the patient through the housing and infusion cannula.

18. The method of claim 17, wherein the second substance stream is injected through the septum by a syringe.

19. The method of claim 17, further comprising:
disconnecting the connecting hub from the housing; and
attaching a plugging system to the housing while leaving the distal end of the infusion cannula within the patient.

20. The system of claim 1, further comprising:
a septum positioned within the housing.

21. The system of claim 20, further comprising:
a funnel shaped guide positioned at a proximal end of the septum.

22. A connecting hub having an internal Y-shaped flow channel structure.

23. The device of claim 22, wherein the internal Y-shaped flow channel structure comprises:
a first flow channel adapted to connect to a proximal end of an infusion cannula;
a second flow channel adapted to connect to a distal end of an infusion delivery tube; and
a third flow channel which is covered by a septum, the first, second and third flow channels all intersecting within the connecting hub.
